(19) United States
(12) Patent Application Publication
(10) Pub. No.: US 2022/0047017 A1
(43) Pub. Date: Feb. 17, 2022

(54) FACE MASKS AND OTHER PROTECTIVE DEVICES HAVING ENHANCED VIRUS ENTRAPMENT EFFICIENCY, AND RELATED METHODS

(71) Applicant: AXIM BIOTECHNOLOGIES, INC., San Diego, CA (US)

(72) Inventors: Sergei A. SVAROVSKY, San Diego, CA (US); John HUEMOELLER, San Diego, CA (US)

(21) Appl. No.: 17/402,355

(22) Filed: Aug. 13, 2021

Related U.S. Application Data
(60) Provisional application No. 63/066,104, filed on Aug. 14, 2020.
(60) Provisional application No. 63/084,407, filed on Sep. 28, 2020.

(51) Int. Cl. A41D 13/11; A61K 38/48

(52) U.S. Cl. CPC A41D 13/1192; A41D 2500/20; A61K 38/4813

(57) ABSTRACT

Protective devices with enhanced entrapment efficiency and related methods are provided herein. Contemplated protective devices include face masks having a filtration material and an infectious-agent-capture-moiety. In some aspects, ACE2 is applied to mask material during the construction of the material it is applied to, during construction of the mask, or after construction of the mask. In some aspects, the infectious agent the infectious-agent-capture-moiety captures is SARS-CoV-2.

### CROSS-REFERENCE TO RELATED APPLICATIONS

This application claims priority to U.S. Provisional Patent application No. 63/066,104, filed Aug. 14, 2020, and U.S. Provisional Patent application No. 63/084,407, filed Sep. 28, 2020. All references, including publications, patent applications, and patents, cited herein are hereby incorporated by reference to the same extent as if each reference were individually and specifically indicated to be incorporated by reference and were set forth in its entirety herein.

### BACKGROUND

### Field of the Invention

Facemasks and other protective devices with enhanced virus entrapment efficiency, and methods of manufacturing the same are provided herein.

### Description of the Related Art

Known face masks act by trapping viruses containing aerosol particles by non-specific absorption on passage through interwoven filter fibers. Some of the drawbacks of known face masks include low filtration efficiency (e.g., up to about 85%), and for virus trapped on or in the face mask, the survival time of viruses on or in the face masks (e.g., several days).

### SUMMARY

Face masks and other protective devices with improved virus entrapment efficiency are provided herein. The entrapment efficiency can be improved via application of highly active receptors for virus spike proteins on or through the mask materials, and can be easily modulated by the amount of the applied receptor, which could be useful depending on the contemplated application (e.g., healthcare, police, airlines).

In one aspect, a protective device comprises a filtration material and an infectious-agent-capture-moiety. As used herein, the term “filtration material” should be interpreted broadly to include any substance that can act as a substrate or scaffold to at least one of: directly block or impede the flow-through of an infectious agent; and to bind, carry or otherwise support an infectious-agent-capture-moiety, which infectious-agent-capture-moiety then functions to directly block or impede the flow-through of an infectious agent. Suitable filtration materials are well-known in the art and include 3-ply material, 5-ply material, N95 material, surgical mask material, any clothing/textile material (e.g, cotton, polyester, wool), a combination thereof, etc. As used herein, the phrase “infectious-agent-capture-moiety” refers to any component or compound or biomolecule that can bind to any infectious agent. In particular embodiments, the infectious-agent-capture-moiety can be selected from: a cell-surface receptor (e.g., angiotensin converting enzyme 2 (ACE2)) or fragment thereof, a modified ACE2 such as those described in U.S. Provisional Application Ser. No. 63/081811, filed Sep. 22, 2020 (which is incorporated herein by reference), high affinity antibody or fragment thereof, nanobody or fragment thereof, a protein receptor such as CD209c and Clec4 (https://www.embopress.org/doi/abs/10.15252/embj.2021108375), a glycan-based receptor such as galactose, N-acetylgalactosamine, N-acetylneuraminic acid (e.g., Neu5NAc, Neu9NAc), heparin, fibronectin, or combinations thereof. In particular embodiments, the antibody is high affinity and is selected from a polyclonal antibody (pAb), a monoclonal antibody (mAb), bi-specific antibody, multi-specific antibody, or the like.

The protective device can comprise a face mask, and the filtration material can optionally be pre-treated with, for example, 20%, 30%, 40%, 50%, 60%, 70%, between 20-70%, between 50-70% (v/v) isopropanol prior to application of the infectious-agent-capture-moiety. Additionally or alternatively, pre-treatment of the filtration material can involve aqueous mixtures with ethanol, propanol, propanediol, isopropanol, butanol, isobutanol, glycerol, acetonitrile, evaporable and non-toxic organic solvent(s), or any combination thereof. An aqueous mixture of water-miscible organic solvent advantageously wets the hydrophobic surface of the mask material to allow for an even distribution of protein (e.g. ACE2) from aqueous or semi-aqueous solutions. Without prewetting, the aqueous solution of protein can deposit in uneven patches. Even capture protein distribution is critical for the mask functionality. In principle, a protein could be deposited from pre-wetting solution directly. The problem is that proteins normally do not tolerate high concentrations of organic solvents used in pre-wetting step, and ACE2 can be completely inactive in 30-70% isopropanol. Thus, Applicant's two-step process can advantageously be used: (1) prewetting in organic solvent/water (e.g., 70% organic solvent/water), and (2) protein deposition from low (e.g., 10-20%) organic solvent/water solution.

In some embodiments, the infectious-agent-capture-moiety composes a solution with a concentration of between 0.1-100, between 10-30, between 30-100, between 15-25, or between 0.5-25 μg/mL of the infectious-agent-capture-moiety. In some embodiments, the infectious-agent-capture-moiety composes an aerosol with a concentration of between 0.1-100, between 10-30, between 30-100, between 15-25, or between 0.5-25 μg/mL of the infectious-agent-capture-moiety. In some embodiments, the infectious-agent-capture-moiety is at least one of a SARS-CoV-2-capture-moiety and a SARS-CoV-2-spike-protein capture-moiety, and the infectious agent is SARS-CoV-2.

As used herein, the phrase “SARS-CoV-2-capture-moiety” refers to any component or compound or biomolecule that can bind to any region of SARS-CoV-2. In particular embodiments, the SARS-CoV-2-capture-moiety can be, as set forth above, selected from the group: ACE2 or fragment thereof, high affinity antibody or fragment thereof, nanobody or fragment thereof. In some embodiments, the antibody is high affinity and is selected from a pAb, a mAb, bi-specific antibody, multi-specific antibody, or the like. As used herein, the phrase “SARS-CoV-2-spike-protein capture-moiety” refers to any component or compound or biomolecule that can bind to any region of SARS-CoV-2-spike-protein. In certain embodiments, the SARS-CoV-2-spike-protein capture moiety is selected from the group: ACE2 or fragment thereof, antibody or fragment thereof, nanobody or fragment thereof. In some embodiments, the antibody is high affinity and is selected from a pAb, a mAb, bi-specific antibody, multi-specific antibody, or the like.

In some embodiments, the infectious-agent-capture-moiety can comprise a SARS-CoV-2-capture moiety, more specifically, a SARS-CoV-2-spike-protein capture-moiety, which can comprise a synthetic antibody or nanobody, such as AeroNabs.

In another aspect, a method of manufacturing a face mask having enhanced infectious agent capturing properties is provided. The method can comprise a step of treating a first filtration material layer with a first solution, and a step of applying an infectious-agent-capture-moiety to the first filtration material layer treated with the first solution to form an enhanced first layer. In some embodiments, the step of treating the first filtration material layer with the first solution may be skipped. It is contemplated that the infectious-agent-capture-moiety may be applied at any time and to any material of a protective device. For example, the infectious-agent-capture-moiety may be applied may be applied during the manufacture of the filter material, or after a face mask is completely assembled.

In another aspect, a method of manufacturing a face mask having enhanced infectious agent capturing properties comprises: applying an infectious-agent-capture-moiety to a first filtration material layer to form an enhanced first layer; mechanically coupling a second material layer to a first side of the enhanced first layer; and mechanically coupling a third material layer to a second side of the enhanced first layer. Contemplated methods can comprise a step of treating the first filtration material layer with an isopropanol solution prior to applying the infectious-agent-capture-moiety.

In some embodiments, the infectious-agent-capture-moiety composes a solution with a concentration of between 0.1-100, between 10-30, between 30-100, between 15-25, or between 0.5-25 μg/mL of the infectious-agent-capture-moiety. In some embodiments, the infectious-agent-capture-moiety composes an aerosol with a concentration of between 0.1-100, between 10-30, between 30-100, between 15-25, or between 0.5-25 μg/mL of the infectious-agent-capture-moiety. In some embodiments, the infectious-agent-capture-moiety is at least one of a SARS-CoV-2-capture-moiety and a SARS-CoV-2-spike-protein capture-moiety, and the infectious agent is SARS-CoV-2. In some embodiments, the infectious-agent-capture-moiety can comprise a SARS-CoV-2- capture moiety, more specifically, a SARS-CoV-2-spike-protein capture-moiety, which can comprise a synthetic antibody.

The infectious-agent-capture-moiety (and optionally the isopropanol) may be applied to additional material layers of the face mask, including inner and outer layer(s). In some embodiments, the infectious-agent-capture-moiety may only be applied to a non-mid-layer. In some embodiments, the infectious-agent-capture-moiety may only be applied to a mid-layer (e.g., mid-layer of 3-ply mask, or a second, third or fourth material of a 5-ply mask).

In some embodiments, the infectious-agent-capture-moiety may be applied to a non-mid-layer and a mid-layer.

Other advantages and benefits of the disclosed assemblies, components and methods will be apparent to one of ordinary skill with a review of the following detailed description.

### BRIEF DESCRIPTION OF THE DRAWINGS

The details of embodiments of the present disclosure, both as to their structure and operation, can be gleaned in part by study of the accompanying drawings, in which like reference numerals refer to like parts, and in which:

FIG. 1 shows layers of a 3-ply face mask having a solution comprising an infectious-agent-capture-moiety sprayed thereon;

FIG. 2 shows an ELISA 96 well plate with mask material cut into 3 mm circular pieces;

FIG. 3 is an ELISA experiment schematic;

FIG. 4 is a table showing the ELISA layout;

FIG. 5 shows the ELISA plate development;

FIGS. 6A-6C illustrate surgical mask results corresponding to the experiment of FIGS. 2-5;

FIGS. 7A-7B illustrate K95 mask results corresponding to the experiment of FIGS. 2-5;

FIG. 8 is a schematic of modifying a fluorescent bead at the surface with virus RBD protein (artificial virus);

FIG. 9 is a schematic of direct RBD-Bead binding experiment;

FIG. 10 shows wettability properties of a mask filter layer;

FIG. 11 shows wettability properties of an outside layer of a mask;

FIG. 12 shows images of mask filter under black light revealing RBD binding patterns.

### DETAILED DESCRIPTION

After reading this description, it will become apparent to one skilled in the art how to practice the claims in various alternative embodiments and alternative applications. However, although various embodiments will be described herein, it is understood that these embodiments are presented by way of example and illustration only, and not limitation. As such, this detailed description of various embodiments should not be construed to limit the scope or breadth of the appended claims.

In one aspect, a protective device (e.g., face mask) comprises a filtration material and an infectious-agent-capture-moiety. In another aspect, a method of manufacturing a protective device comprises applying an infectious-agent-capture-moiety to the first filtration material layer, and optionally pre-treating the first filtration material layer with a first solution (e.g., isopropanol) prior to applying the infectious-agent-capture-moiety. In another aspect, a method of manufacturing a protective device comprises applying an infectious-agent-capture-moiety to a first filtration material layer to form an enhanced first layer, mechanically coupling a second material layer to a first side of the enhanced first layer, and mechanically coupling a third material layer to a second side of the enhanced first layer.

The infectious-agent-capture-moiety can be applied via a solution or aerosol of between 0.1-100, between 10-30, between 30-100, between 15-25, or between 0.5-25 μg/mL of the infectious-agent-capture-moiety. In some embodiments, the infectious-agent-capture-moiety is aerosolized and sprayed or applied onto pretreated filtration material. In other embodiments, the aerosolized infectious-agent-capture-moiety is diluted (in some embodiments, extremely diluted; e.g. 0.1 μg/ml diluted) prior to being sprayed or applied to the filtration material. In other embodiments, suitable dilutions of the aerosolized infectious-agent-capture-moiety prior to spraying or application the filtration material can be selected from: 0.9 μg/ml diluted, 0.8 μg/ml diluted, 0.7 μg/ml diluted, 0.6 μg/ml diluted, 0.5 μg/ml diluted, 0.4 μg/ml diluted, 0.3 μg/ml diluted, 0.2 μg/ml diluted, 0.1μg/ml diluted, 0.09 μg/ml diluted, 0.08 μg/ml diluted, 0.07 μg/ml diluted, 0.06 μg/ml diluted, 0.05 μg/ml diluted, 0.04 μg/ml diluted, 0.03 μg/ml diluted, 0.02 μg/ml diluted, 0.01 μg/ml diluted, or less, and the like.

In some embodiments, when multi-layer masks are used (e.g., well-known 3-ply and 5-ply masks), the middle layer of the filtration material can be sprayed. In accordance with the present invention, the face masks provide enhanced protection for the user from infection by infectious agents, such as SARS-CoV-2, in addition to enhanced protection of others. In one embodiment, for example, 1 mg of infectious-agent-capture-moiety (e.g., ACE2) will produce 10 liters of aerosol, which is then sprayed or applied to the filtration material. The infectious-agent-capture-moiety can be applied to the filtration material at any time, such as during the construction of the face masks, or during the construction of the filtration material itself. Accordingly, also provided herein in accordance with the present invention, is an enhanced-filtration-material comprising a substrate or scaffold; and infectious-agent-capture-moiety (e.g., ACE2) connected to the substrate or scaffold. For example, during or after the production of cotton, polyester, wool or the like, the aerosolized infectious-agent-capture-moiety is sprayed or applied to the cotton, polyester, wool, or the like, such that the material can later be used in the construction of face masks with enhanced protective properties.

The infectious-agents captured in accordance with the present invention are typically those that are airborne (e.g., SARS-CoV-2, coronavirus, flu virus, ebola). In some embodiments, the infectious agent is SARS-Cov-2. Coronavirus-neutralizing antibodies primarily target the trimeric spike (S) glycoproteins on the viral surface that mediate entry into host cells. The S protein has two functional subunits that mediate cell attachment (the S1 subunit, existing of four core domains S1A through S1D) and fusion of the viral and cellular membrane (the S2 subunit). Potent neutralizing antibodies often target the receptor interaction site in S1, disabling receptor interactions. In certain embodiments related to the SARS-CoV-2 infection agent, numerous anti-SARS-CoV-2 antibody capture-moieties are known and currently poised to enter clinical trials during the summer of 2020. Therapeutic trials will include treatment of patients with SARS-CoV-2 infection, with varying degrees of illness, to block disease progression. These antibodies include, e.g., LY-CoV555, a mAb isolated from a recovered COVID-19 patient (Eli Lilly); Regeneron's investigational double mAb combination, REGN-COV-2, which is designed to bind to two points on the SARS-CoV-2 spike protein; the human 47D11 antibody that binds to cells expressing the full-length spike proteins of SARS-CoV and SARS-CoV-2 (as described in Wang et al., Nature Communications, Volume 11, Article number: 2251 (2020); each of which is incorporated herein by reference in its entirety for all purposes).

In some embodiments, the SARS-CoV-2-capture-moiety or SARS-CoV-2-spike-protein capture-moiety is an AeroNab (available from UCSF). These synthetic antibodies, referred to as AeroNabs, can be administered as a nasal spray to protect people from coronavirus. These aerosolized agents known as Aeronab's trace back to a tiny molecule first discovered in camels and similar animals, called a nanobody. They are smaller than human antibodies, and can be manipulated to perform specific tasks, such as in accordance with the present invention, attaching themselves to the spike proteins on the coronavirus. It has been found that the antiSARS-Cov-2 AeroNabs bind to one of the spike proteins and never lets go. SARS-CoV-2 spike proteins fits into ACE2 receptor of lung cell. This allows it to enter the cells. Coronavirus infection is only possible if the spike protein is allowed to interact with ACE2. Synthetic nanobodies (tiny antibodies) (AeroNab) bind well to spike proteins so the virus can't attach to ACE2. It is aerosolized and can be self-administered via an inhaler or nasal spray. When AeroNabs bind to the spike protein, the virus cannot pass through the face masks contemplated herein and thus loses its ability to infect the covered and protected individual. The AeroNabs are stable enough to be turned into an effective aerosol. In particular embodiments, the AeroNabs nasal spray can be sprayed or applied directly to the filtration materials of the face masks contemplated herein.

### Example 1

Currently available masks act by trapping the virus containing aerosol particles by non-specific absorption on passage through interwoven filter fibers. Non-specific absorption is not 100% efficient, and the filtration efficiency of ordinary filtering materials can only reach up to 85%. Moreover, virus entrapped on the surfaces continues to be viable 24-48 hours (or longer) post-capture. In an attempt to address this problem, some companies developed chemically treated masks (citrate, zinc/copper) that speed up virus disintegration after entrapment. However, this doesn't solve the problem of the entrapment efficiency. The face masks of the inventive subject matter improve virus entrapment efficiency by applying highly active receptor for the virus spike protein to the mask materials, as shown by the experiments described herein. The entrapment efficiency can be easily modulated by the amount of the applied receptor, which could be useful depending on how the masks will be used (e.g., by healthcare workers, by non-essential workers, by restaurant workers, by police, by firefighters).

Surgical masks generally comprise a sandwich of an outer and inner layer of spunbond fabric, and a mid-layer of melt-blown fabric. The outer layer typically comprises hydrophobic spunbond fabric, and the inner layer typically comprises a soft absorbent spunbond fabric. In some aspects, a virus specific ACE2 receptor or other infectious-agent-capture-moiety is applied to the melt-blown polypropylene fibers of the mid-layer.

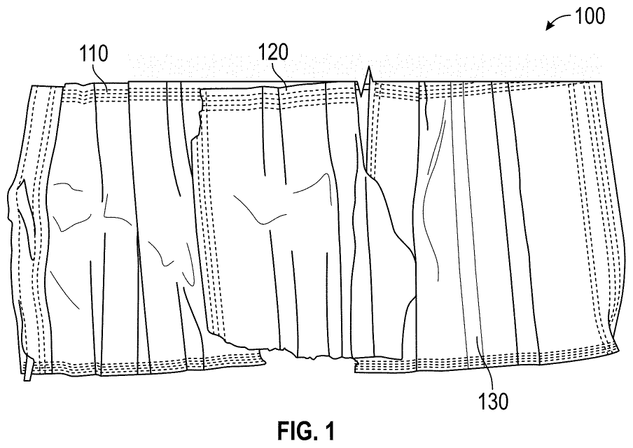

As shown in FIG. 1, three concentrations (1, 10, and 100 μg/mL) of ACE2 were aerosol-dispensed on each side of the three layers (outside layer 110, mid-layer 120, inner layer 130) of a 3-ply face mask 100. The same was done with a K-95 mask.

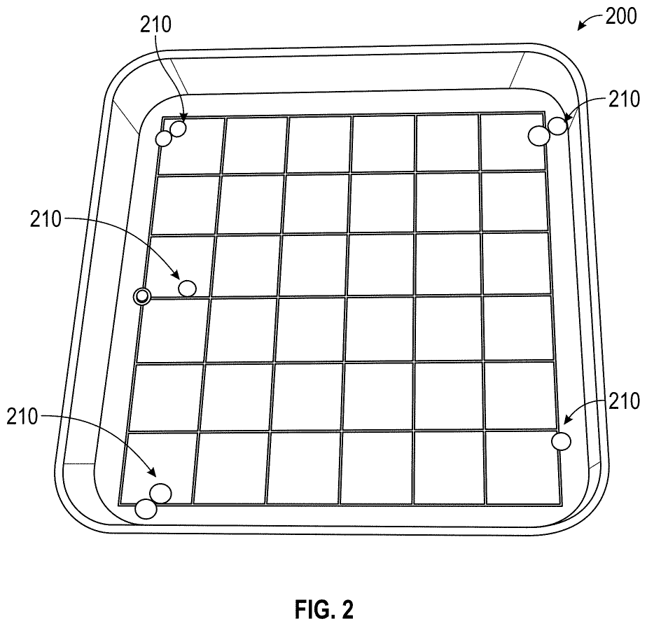

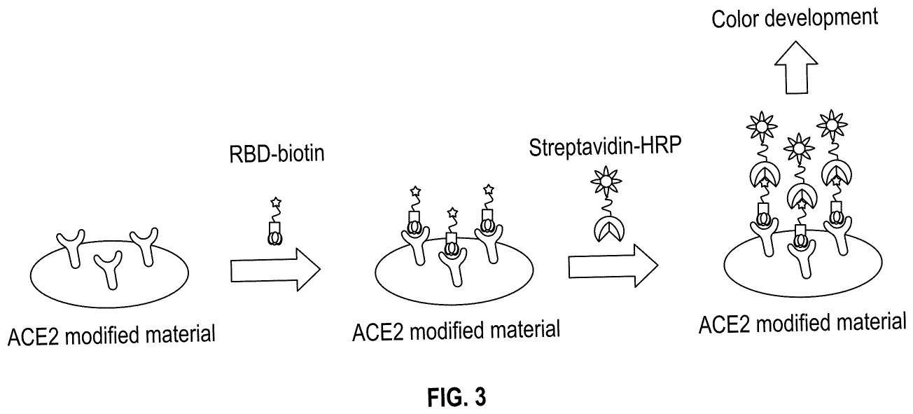

As shown in FIG. 2, each striped material was cut into 3 mm circular pieces and placed into each well of ELISA 96 well plate in duplicate. FIG. 3 shows the ELISA experiment schematic. SARS-CoV-2 virus enters human cells via ACE2 receptors via the interaction of the receptor binding domain (RBD) of the spike protein on the viral surface. Here, ACE2 modified mask material is placed in the 96 well plate. The mask material was first blocked with an ELISA blocking buffer (2% BSA in 1×PBS supplemented with 0.05% Tween-20 detergent) for 30 min at 37 C, washed with PBST buffer 3 times and then RBD-biotin (Axim Biotechnologies, Inc) diluted to 10 μg/mL in an ELISA binding buffer (1% BSA in 1×PBS supplemented with 0.025% Tween-20 detergent) was added. The reaction mixture was incubated for 30 min, washed 3 times with PBST (Phosphate Buffered Saline supplemented with 0.025% Tween-20 detergent) and then HRP-conjugated streptavidin (Axim Biotechnologies, Inc) was added. The streptavidin binds to biotin and the conjugated HRP provides enzyme activity for detection with TMB (3,3′,5,5′-tetramethylbenzidine) substrate (Seracare Life Sciences, Inc).

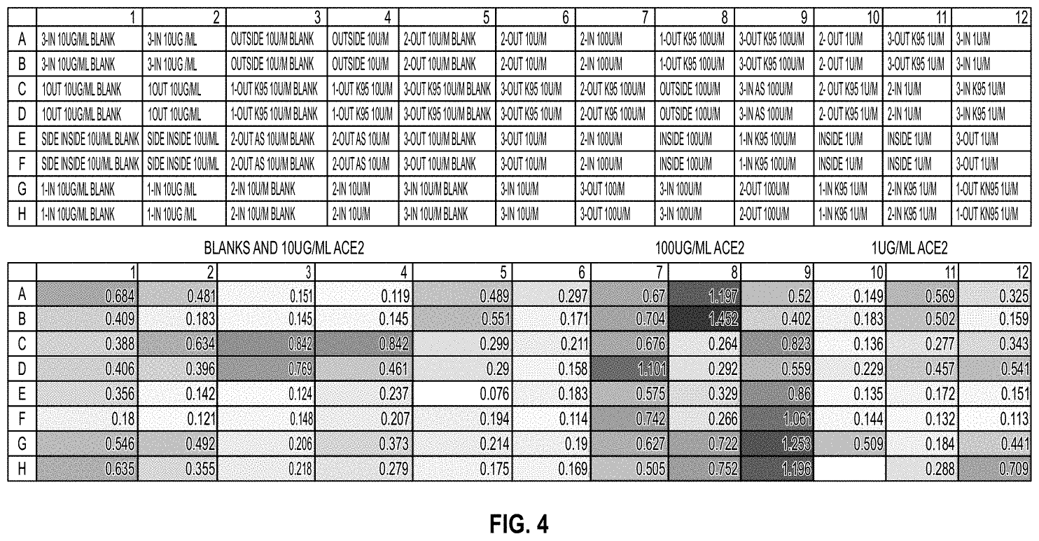

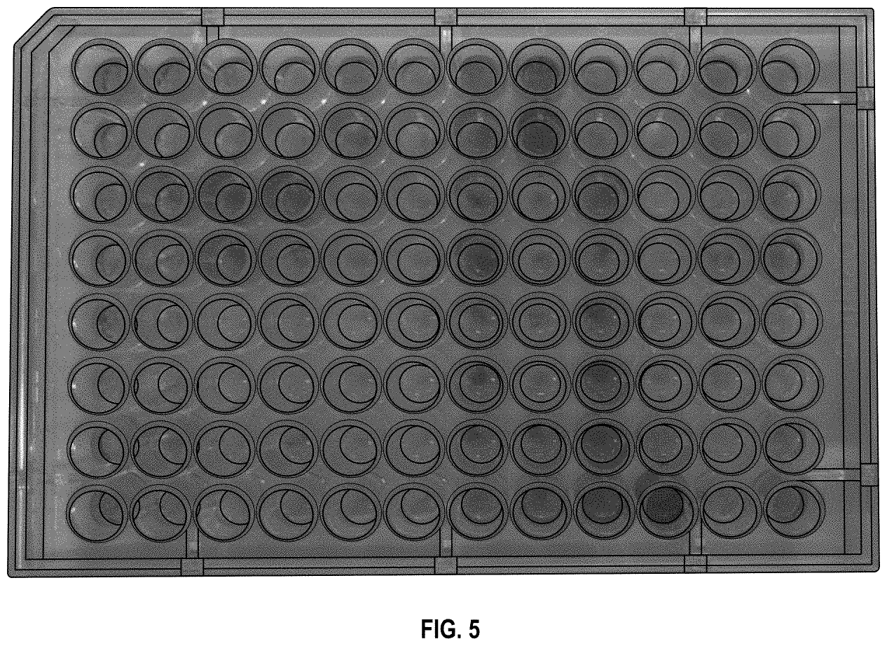

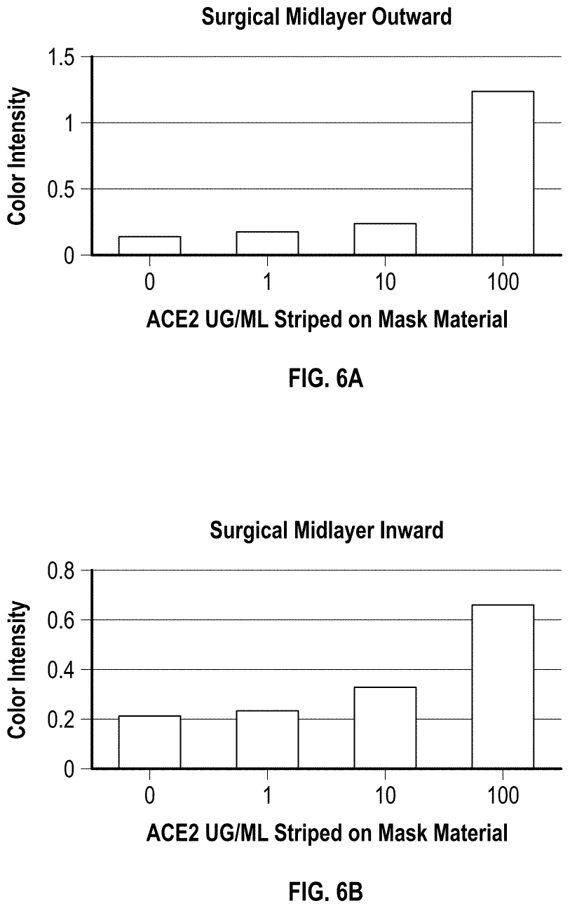

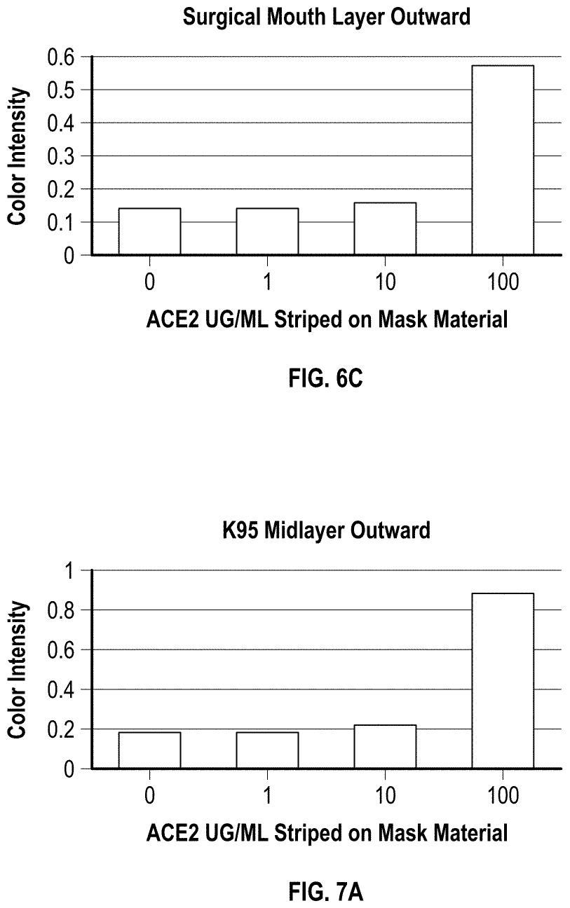

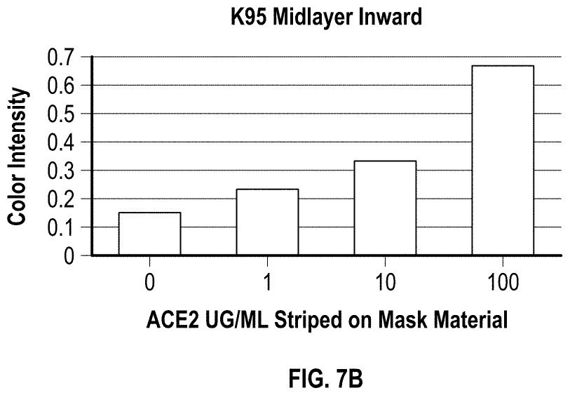

FIG. 4 is a table showing the ELISA layout (wherein 1=inner layer, 2=middle layer filter, and 3=outer layer, and “in” refers to facing inside the mask, and “out” refers to facing outside). FIG. 5 shows an image of the ELISA plate development. As shown in FIGS. 6A-6C, ACE2 gets immobilized onto surgical mask filter material and ACE2 is actively binding RBD, with increased color intensity corresponding to increased ACE2 concentration and increased ACE2-RBD binding. As shown in FIGS. 7A-7B, ACE2 gets immobilized onto K95 mask filter material and ACE2 is actively binding RBD, with greater color intensity corresponding to increased ACE2 concentration and increased ACE2-RBD binding.

### Example 2

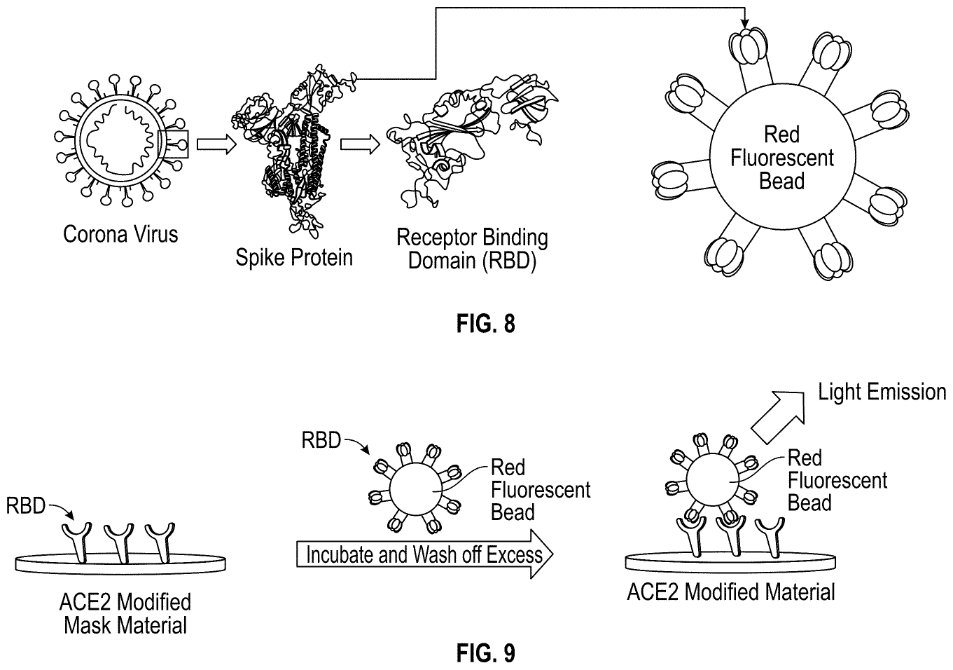

Particle conjugated with RBD protein acts as a safe surrogate of coronavirus that can be tracked in real time, (see FIG. 8). As illustrated in FIG. 9, ACE2 protein receptor (Axim Biotechnologies, Inc) gets impregnated into mask filter material. Fluorescent bead (Bangs Laboratories, Inc) modified at the surface with virus RBD protein (artificial virus) (Axim Biotechnologies, Inc) is applied to the mask material. Immobilized ACE2 captures the bead and now can be visualized by light emission. The mask acts as a decoy material for the virus, and the virus gets permanently entangled into mask material.

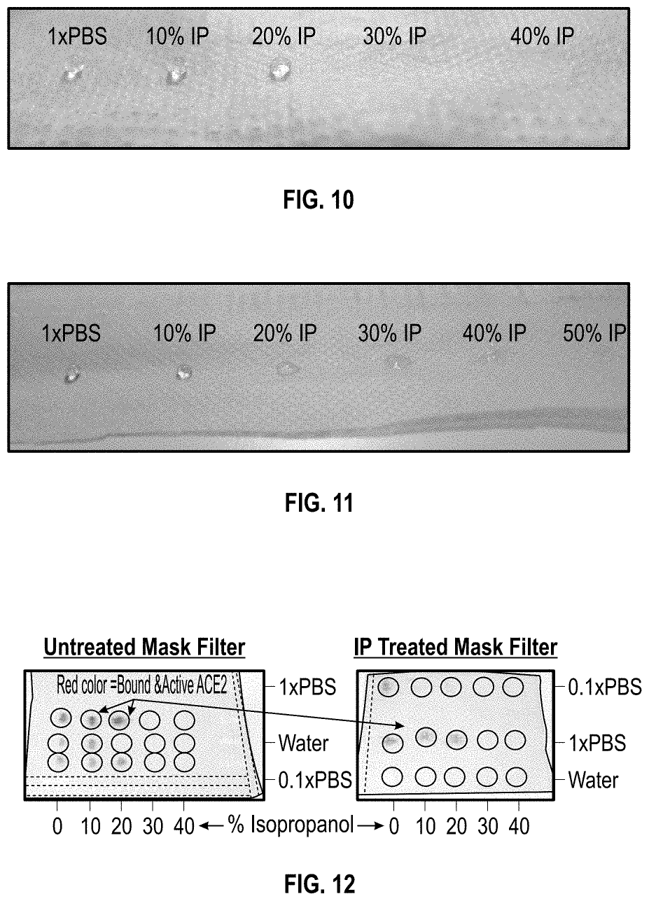

FIG. 10 shows the results of investigating wettability properties of the polypropylene based mask filter by applying droplets of variously diluted isopropanol in Phosphate Buffered Saline (PBS) solution. Isopropanol is a hydrophobic alcohol that is fully miscible with water. It has affinity to stick to hydrophobic (water-repelling) surfaces. As shown in FIG. 10, contact angle (spread) is reduced with increasing isopropanol concentrations. At 0-20% isopropanol, the solution does not penetrate through the filter. At >20% isopropanol may affect ACE2 structural integrity. FIG. 11 shows wettability properties of an outside layer of a mask. The outside layer is made from the same material and behaves similarly to the filter mid-layer.

One mask filter was left untreated (as is), and a second was first treated with 70% isopropanol (IP) and then blotted between paper towels to remove excess. Five solutions of 20 μg/mL ACE2 (Axim Biotechnologies, Inc) were prepared in 0, 10, 20, 30 and 40% isopropanol diluted with either water, 0.1×PBS, or 1×PBS. 3 μL of the ACE2 solutions were applied directly on the surface of the masks. Mask filters were dried at 37 degrees C. for 30 minutes and then overnight at room temperature over desiccant bags. The mask filters were blocked with ELISA blocking buffer for 30 minutes, washed 2×10 min with PBST. Mask filters were incubated for 30 min with a solution of 10 μg/mL fluorescent beads conjugated with RBD and diluted in ELISA buffer, then washed 2×10 min with PBST. Imaging under black light revealed the RBD binding patterns shown in FIG. 12, indicating that (a) protein is attached, and (b) is actively binding RBD. A negative control, untreated mask, material showed no binding with RBD beads under identical conditions.

Results. Deposition from 30% and above isopropanol did not result in stable deposition in all cases. Deposition on untreated mask resulted in hard surface deposition where the ACE2 protein is attached instantly at the place of deposition thus providing no dispersive deposition. Deposition on treated mask in 10-20% isopropanol gave complete penetration of ACE2 throughout the fibers. Deposition was always better from 1×PBS (salt). No deposition was observed on treated mask from water/isopropanol mixture. Thus, it is contemplated that a protocol of the inventive subject matter involves pre-treatment with about 70% isopropanol and spraying ACE2 from 10-20% isopropanol/1×PBS mixture.

Thus, specific protective devices, components thereof, and methods of manufacturing protective devices with enhanced virus entrapment efficiency have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

As used herein, and unless the context dictates otherwise, the term “coupled to” is intended to include both direct coupling (in which two elements that are coupled to each other contact each other) and indirect coupling (in which at least one additional element is located between the two elements).

The recitation of ranges of values herein is merely intended to serve as a shorthand method of referring individually to each separate value falling within the range, and including the endpoints. Unless otherwise indicated herein, each individual value is incorporated into the specification as if it were individually recited herein.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “assembly,” “mechanism,” “element,” “device,” and the like may not be a substitute for the word “means.” As such, no claim element is to be construed as a means plus function unless the element is expressly recited using the phrase “means for.”

Various modifications to the embodiments described herein will be readily apparent to those skilled in the art, and the general principles described herein can be applied to other embodiments without departing from the spirit or scope of the claims. Thus, it is understood that the scope of the claims fully encompasses other embodiments that may become obvious to those skilled in the art and that the scope of the claims are accordingly not limited.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

What is claimed is:

1. A protective device, comprising:
a filtration material; and
an infectious-agent-capture-moiety.

2. The protective device of claim 1, wherein the protective device is a face mask.

3. The protective device of claim 2, wherein the filtration material is a pre-treated filtration material.

4. The protective device of claim 2, wherein the infectious-agent-capture-moiety comprises a cell-surface receptor or fragment thereof.

5. The protective device of claim 4, wherein the cell-surface receptor is ACE2, and wherein the infectious-agent-capture-moiety can bind to SARS-CoV-2.

6. The protective device of claim 5, wherein the infectious-agent-capture-moiety composes a solution with a concentration of between 0.5-25 μg/mL of the infectious-agent-capture-moiety.

7. The protective device of claim 5, wherein the infectious-agent-capture-moiety composes an aerosol with a concentration of 0.5-25 μg/mL of the infectious-agent-capture-moiety.

8. The protective device of claim 1, wherein the infectious-agent-capture-moiety is a SARS-CoV-2 capture-moiety.

9. The protective device of claim 8, wherein the SARS-CoV-2 capture-moiety is a SARS-CoV-2-spike-protein capture-moiety.

10. The protective device of claim 9, wherein the SARS-CoV-2-spike-protein capture-moiety comprises a synthetic antibody.

11. A method of manufacturing a face mask having enhanced infectious agent capturing, comprising:
applying an infectious-agent-capture-moiety to a first filtration material layer to form an enhanced first layer;
mechanically coupling a second material layer to a first side of the enhanced first layer; and
mechanically coupling a third material layer to a second side of the enhanced first layer.

12. The method of claim 11, further comprising applying the infectious-agent-capture-moiety to at least one of the second and third material layers.

13. The method of claim 11, further comprising treating the first filtration material layer with an isopropanol solution prior to applying the infectious-agent-capture-moiety.

14. The method of claim 11, wherein the infectious-agent-capture-moiety composes a solution with a concentration of between 0.5-25 μg/mL of the infectious-agent-capture-moiety.

15. The method of claim 11, wherein the infectious-agent-capture-moiety composes an aerosol with a concentration of 0.5-25 μg/mL of the infectious-agent-capture-moiety.

16. The method of claim 11, wherein infectious-agent-capture-moiety is ACE2.

17. The method of claim 11, wherein the infectious-agent-capture-moiety is a SARS-CoV-2 capture-moiety.

18. The method of claim 17, wherein the SARS-CoV-2 capture-moiety is a SARS-CoV-2-spike-protein capture-moiety.

19. The method of claim 11, wherein the infectious-agent-capture-moiety comprises a synthetic antibody.

20. A method of manufacturing a face mask having enhanced infectious agent capturing, comprising:
treating a first filtration material layer with a first solution; and
applying an infectious-agent-capture-moiety to the first filtration material layer treated with the first solution to form an enhanced first layer.
